(19) United States
(12) Patent Application Publication
(10) Pub. No.: US 2011/0015536 A1
(43) Pub. Date: Jan. 20, 2011

(54) EEG-based method for determining a subject's compatibility with a work environment

(76) Inventors: Michael Milgramm, Valley Stream, NY (US); Alex Imas, Niles, IL (US)

Correspondence Address: COOPER & DUNHAM, LLP, 30 Rockefeller Plaza, 20th Floor, NEW YORK, NY, 10112, US

(21) Appl. No.: 12/460,395

(22) Filed: Jul. 17, 2009

(51) Int. Cl. A61B 5/0484

(52) U.S. Cl. 600/544

(57) ABSTRACT

This invention provides an EEG-based method of determining whether a test subject possesses an attitude compatible with the work environment at a predetermined organization.

### BACKGROUND OF THE INVENTION

It is a commonly known fact that when one places two conducting electrodes connected to a voltmeter, one on the scalp and the other on an electrically neutral area, such as the mastoids behind the ears, a quantifiable voltage can be observed. This voltage signal and its change with respect to time is the basis of electroencephalography, or EEG. The signal measured on the scalp is actually a summation of individual postsynaptic potentials occurring within the brain. Since both the neural tissue and the skull act as a low pass filter, it is unlikely that the high frequency transients of action potentials would make it up to the scalp, and since postsynaptic potentials generally have lower frequency transients associated with them, it is widely believed that the observed EEG signal originates from them.

The EEG recording is characterized by amplitude, frequency and their change over time. The frequency component of the EEG can be utilized to infer the level of an individual's neural activity. The frequencies are broken down into ranges which describe how alert and conscious a person is at any given time. The delta frequency (1-4 Hz) is associated with deep sleep. The theta frequency (5-7 Hz) is associated with drowsiness, and delta activity is also common. The alpha frequency (8-13 Hz) is associated with relaxed wakefulness, where not much brain resources are devoted to any one thing. The beta frequency (12-20 Hz, or 30 Hz) and the gamma frequency (36-44 Hz) are associated with alert attentiveness.

The technology disclosed herein uses EEG analysis to screen an individual's compatibility with a particular organization, regardless of the individual's spoken answers to interview questions and other screening procedures.

### SUMMARY OF THE INVENTION

A method of determining whether a test subject possesses an attitude compatible with employment at a predetermined organization comprising:

a) recording a first electroencephalograph (EEG) from the test subject over a first period of time and performing a power component analysis on brain wave activity from the EEG of the test subject so as to thereby determine if the subject is alert enough to proceed to step b) of the method, and if so, proceeding to step b);

b) recording a second EEG from the test subject over a second period of time and exposing the test subject during the second period of time to a series of visual images, which images have been predetermined to be emotionally neutral in a reference population of subjects, and quantitating the levels of alpha and beta wave activity in the test subject during the second period of time so as to determine baseline alpha and beta wave activity levels;

c) recording a third EEG over a third period of time from the test subject and exposing the test subject during the third period of time to a series of pairs of sensory stimuli, wherein the pairs each consist of a visual stimulus and a corresponding auditory stimulus, and quantitating the levels of alpha and beta wave activity in the test subject during exposure of the subject to the series of pairs of sensory stimuli;

d) subtracting the baseline levels of alpha and beta wave activity quantitated during step b) from the levels of alpha and beta wave activity, respectively, quantitated during step d), so as to determine corrected alpha wave activity and corrected beta wave activity in the subject;

e) determining the ratio of corrected alpha wave activity to corrected beta wave activity,

f) determining if the ratio of corrected alpha wave activity to corrected beta wave activity is higher in the RCH of the test subject or in the LCH of the test subject,

wherein a higher alpha/beta ratio in the RCH than the LCH indicates that the test subject has an attitude compatible with employment at the predetermined organization, and wherein a higher alpha/beta ratio in the LCH than the RCH indicates that the test subject does not possesses an attitude compatible with employment at the predetermined organization.

A method of determining whether a test subject possesses an attitude compatible with performing work for a predetermined organization comprising:
a) recording a first electroencephalograph (EEG) from the test subject over a first period of time and performing a power component analysis on brain wave activity from the EEG of the test subject so as to thereby determine if the subject is alert enough to proceed to step b) of the method, and if so, proceeding to step b);
b) recording a second EEG from the test subject over a second period of time and exposing the test subject during the second period of time to a series of visual images, which images have been predetermined to be emotionally neutral in a reference population of subjects, and quantitating the levels of alpha and beta wave activity in the test subject during the second period of time so as to determine baseline alpha and beta wave activity levels;
c) recording a third EEG over a third period of time from the test subject and exposing the test subject during the third period of time to a series of pairs of sensory stimuli, wherein the pairs each consist of a visual stimulus and a corresponding auditory stimulus, and quantitating the levels of alpha and beta wave activity in the test subject during exposure of the subject to the series of pairs of sensory stimuli;
d) subtracting the baseline levels of alpha and beta wave activity quantitated during step b) from the levels of alpha and beta wave activity, respectively, quantitated during step d), so as to determine corrected alpha wave activity and corrected beta wave activity in the subject;
e) determining the ratio of corrected alpha wave activity to corrected beta wave activity,
determining if the ratio of corrected alpha wave activity to corrected beta wave activity is higher in the RCH of the test subject or in the LCH of the test subject,
   wherein a higher alpha/beta ratio in the RCH than the LCH indicates that the test subject has an attitude compatible with performing work for the predetermined organization, and wherein a higher alpha/beta ratio in the LCH than the RCH indicates that the test subject does not possesses an attitude compatible with performing work for the predetermined organization.

### BRIEF DESCRIPTION OF THE FIGURES

FIG. 1: Process for collecting EEG information.

FIG. 2: Pin electrode EEG headcap device including an optional screen (e.g. video) for presenting visual images.

FIG. 3: Top view of Pin electrode EEG headcap including an optional screen (e.g. video) for presenting visual images.

FIG. 4: Emotional quadrants schematic.

### DETAILED DESCRIPTION OF THE INVENTION

A method of determining whether a test subject possesses an attitude compatible with employment at a predetermined organization comprising:

a) recording a first electroencephalograph (EEG) from the test subject over a first period of time and performing a power component analysis on brain wave activity from the EEG of the test subject so as to thereby determine if the subject is alert enough to proceed to step b) of the method, and if so, proceeding to step b);

b) recording a second EEG from the test subject over a second period of time and exposing the test subject during the second period of time to a series of visual images, which images have been predetermined to be emotionally neutral in a reference population of subjects, and quantitating the levels of alpha and beta wave activity in the test subject during the second period of time so as to determine baseline alpha and beta wave activity levels;

c) recording a third EEG over a third period of time from the test subject and exposing the test subject during the third period of time to a series of pairs of sensory stimuli, wherein the pairs each consist of a visual stimulus and a corresponding auditory stimulus, and quantitating the levels of alpha and beta wave activity in the test subject during exposure of the subject to the series of pairs of sensory stimuli;

d) subtracting the baseline levels of alpha and beta wave activity quantitated during step b) from the levels of alpha and beta wave activity, respectively, quantitated during step d), so as to determine corrected alpha wave activity and corrected beta wave activity in the subject;

e) determining the ratio of corrected alpha wave activity to corrected beta wave activity,

f) determining if the ratio of corrected alpha wave activity to corrected beta wave activity is higher in the RCH of the test subject or in the LCH of the test subject,

wherein a higher alpha/beta ratio in the RCH than the LCH indicates that the test subject has an attitude compatible with employment at the predetermined organization, and wherein a higher alpha/beta ratio in the LCH than the RCH indicates that the test subject does not possesses an attitude compatible with employment at the predetermined organization.

In an embodiment (1) a higher alpha/beta ratio in the RCH than LCH and an alpha/beta ratio in excess of two standard deviations of baseline indicates that the subject is content and/or calm and possesses an attitude compatible with the predetermined organization, and (2) a higher alpha/beta ratio in the RCH than LCH and an alpha/beta ratio below two standard deviations of baseline indicates that the subject is excited and/or happy and possesses an attitude compatible with the predetermined organization.

In an embodiment the first, second and third EEGs are recorded using a bilateral electrode with one pole of the bilateral electrode positioned over the left cerebral hemisphere of the test subjects' brain and with another pole of the bilateral electrode positioned over the right cerebral hemisphere of the test subjects' brain.

In an embodiment in step a) the test subject is determined to be alert by (a) an alpha waveband power component ratio of 0.5-1.0 and a theta waveband power component ratio of less than 0.5 or (b) a theta waveband power ratio of 0.5-1.0 and an alpha waveband power component ratio of less than 0.5.

In an embodiment the EEGs are recorded using electrodes each comprising an Ag-AgCl recording tip. In an embodiment a 3-D reconstruction of the EEG recorded is not performed. In an embodiment no p300 recordings are made from the subject during the first, second and third period of time. In an embodiment the pairs of sensory stimuli consisting of a visual stimulus and a corresponding auditory stimulus are provided by the predetermined organization. In an embodiment the EEGs are recorded by a wireless EEG headset. In an embodiment in step b) the test subject is exposed to a series of two visual images. In an embodiment in step c) the visual image of the pair of stimuli comprises text and an illustration.

A method of determining whether a test subject possesses an attitude compatible with performing work for a predetermined organization comprising:

a) recording a first electroencephalograph (EEG) from the test subject over a first period of time and performing a power component analysis on brain wave activity from the EEG of the test subject so as to thereby determine if the subject is alert enough to proceed to step b) of the method, and if so, proceeding to step b);

b) recording a second EEG from the test subject over a second period of time and exposing the test subject during the second period of time to a series of visual images, which images have been predetermined to be emotionally neutral in a reference population of subjects, and quantitating the levels of alpha and beta wave activity in the test subject during the second period of time so as to determine baseline alpha and beta wave activity levels;

c) recording a third EEG over a third period of time from the test subject and exposing the test subject during the third period of time to a series of pairs of sensory stimuli, wherein the pairs each consist of a visual stimulus and a corresponding auditory stimulus, and quantitating the levels of alpha and beta wave activity in the test subject during exposure of the subject to the series of pairs of sensory stimuli;

d) subtracting the baseline levels of alpha and beta wave activity quantitated during step b) from the levels of alpha and beta wave activity, respectively, quantitated during step d), so as to determine corrected alpha wave activity and corrected beta wave activity in the subject;

e) determining the ratio of corrected alpha wave activity to corrected beta wave activity,

f) determining if the ratio of corrected alpha wave activity to corrected beta wave activity is higher in the RCH of the test subject or in the LCH of the test subject,

wherein a higher alpha/beta ratio in the RCH than the LCH indicates that the test subject has an attitude compatible with performing work for the predetermined organization, and wherein a higher alpha/beta ratio in the LCH than the RCH indicates that the test subject does not possesses an attitude compatible with performing work for the predetermined organization.

In an embodiment no p300 recordings are made from the subject during the first, second and third period of time.

In an embodiment (1) a higher alpha/beta ratio in the RCH than LCH and an alpha/beta ratio in excess of two standard deviations of baseline indicates that the subject is content and/or calm and possesses an attitude compatible with performing work for the predetermined organization, and (2) a higher alpha/beta ratio in the RCH than LCH and an alpha/beta ratio below two standard deviations of baseline indicates that the subject is excited and/or happy and possesses an attitude compatible with performing work for the predetermined organization.

In an embodiment the first, second and third EEGs are recorded using a bilateral electrode with one pole of the bilateral electrode positioned over the left cerebral hemisphere of the test subjects' brain and with another pole of the bilateral electrode positioned over the right cerebral hemisphere of the test subjects' brain.

In an embodiment in step a) the test subject is determined to be alert by (a) an alpha waveband power component ratio of 0.5-1.0 and a theta waveband power component ratio of less than 0.5 or (b) a theta waveband power ratio of 0.5-1.0 and an alpha waveband power component ratio of less than 0.5.

In an embodiment the EEGs are recorded using electrodes each comprising an Ag-AgCl recording tip. In an embodiment a 3-D reconstruction of the EEG recorded is not performed. In an embodiment the pairs of sensory stimuli consisting of a visual stimulus and a corresponding auditory stimulus are provided by the predetermined organization. In an embodiment the EEGs are recorded by a wireless EEG headset. In an embodiment in step b) the test subject is exposed to a series of two visual images.

In an embodiment in step c) the visual image of the pair of stimuli comprises text and an illustration.

As used herein “images have been predetermined to be emotionally neutral in a reference population of subjects” shall mean images that have been determined through EEG studies of a population of subjects to not cause an emotional arousal in the majority of the population. Non-limiting examples include SUCH images as stored in the emotion annotated image library (LAPS) (e.g. see Lang, P. J., Bradley, M. M., & Cuthbert, B. N. (2005). International affective picture system (IAPS): Affective ratings of pictures and instruction manual. Technical Report A-6. University of Florida, Gainesville, Fla.).

A “corresponding” auditory stimulus, with regard to a paired visual stimulus, is an audible stimulus (e.g. a recording of speech) which describes, or is consistent with, the visual image with which it is paired. By pairing, it is understood that the visual image and corresponding auditory stimulus are presented contemporaneously with each other or within such close order that the subject recognizes that the stimuli are associated.

In regard to determining the ratio of corrected alpha wave activity to corrected beta wave activity, the baseline beta and alpha readings form a “normal population” for each individual. The normal population has a certain mean x (which is different for each individual) and a standard deviation value s.d. (which is also different for each individual). Alpha and beta readings will be recorded stimuli are presented to the subject, e.g. the respective images are shown on the screen. These values and ratios are statistically compared to the baseline values and, if they fall 2 s.d. values away from the baseline mean, then they are considered different to a statistically significant degree. An “increase” or “decrease” in alpha/beta activity refers to a statistically significant difference wherein the new value falls 2 standard deviations to the right or left of the baseline mean.

In regard to the power component analysis, a frequency band power is computed through a Power Spectrum Analysis (PSA) wherein a Fast Fourier Transform (FET) is applied to the raw EEG signal and a power spectrum is computed (μV̂2/Hz). The spectrum is then condensed and analyzed into frequency bands divided into delta (1-4 Hz), theta (4-8 Hz), alpha (8-12 Hz) and beta (12-20 Hz) components.

Where a range is given herein it is understood that the range includes all integers and 0.1 units thereof within that range, as well as any sub-range thereof. For example, a range of 30 minutes to 24 hours includes the times 31.1, 31.2, 31.3, 31.4, 31.5, 31.6, 31.7, 31.8, 31.9 minutes, 32 minutes etc., as well as the ranges 45 minutes to 55 minutes, 44 minutes to 59 minutes, etc.

The embodiments described herein may be performed employing a computer and associated relevant apparatus as described herein.

EEG recording and the apparatus that may be used therefor are described in Allison et al., U.S. Patent Application Publication No. 2005/0017870; Preston, U.S. Pat. No. 5,267,570; Gevins, U.S. Pat. No. 5,724,987; Gevins, U.S. Pat. No. 5,447,166; Gevins, U.S. Pat. No. 5,295,491; Maynard, U.S. Pat. No. 5,816,247; Burton, U.S. Patent Application Publication No. 2004/0044293; Levendowski et al., U.S. Pat. No. 6,625,485; Levendowski et al., U.S. Pat. No. 6,496,724; Johnson, U.S. Pat. No. 6,754,524; Moore-Ede, U.S. Pat. No. 6,511,424; Moore-Ede, U.S. Pat. No. 6,070,098; and Pavelka., WO 2006/000166, each of which is hereby incorporated by reference.

All combinations of the various elements described herein are within the scope of the invention.

### Experimental Details

Organizations such as corporations, government agencies and the military operate through a network of individuals each playing a specific role in the overall structure. Usually, individuals are grouped by hierarchy and function in a fashion resembling a tree diagram. Most occupy labor-intensive, subordinate positions, followed by several levels of management and a few positions on top of the tree responsible for the overall performance of the organization. A position on the bottom can typically be traced to a position on the top by following the corresponding “branches”. When hiring new members to either replace with others or add positions to the network, an organization usually attempts to find an individual with the qualifications and experience that best fit the tasks assigned to that position. Unfortunately, what is often disregarded or not discernable in the hiring process is the individual's compatibility with the organization's culture and work environment.

Every organization is structured differently. Management styles differ not only between organizations but also between different departments within a single organization. Hence, when hiring a new member to fill a particular role, it is also vital that the member is able to function effectively in his or her work environment. Interviewers routinely ask potential hires what they can contribute in the context of the job description, but very few questions, if at all, address the compatibility of the person with the group that they will be joining. In addition, even if such questions are asked, the skilled interviewee can present answers that may result in being offered employment, even though the answers may not be truthful. As a result, highly qualified individuals are hired, trained and placed within an organization only to leave or be terminated a short time later because they simply do not function well in the given environment. Both the organization and the individual lose money and time in the failed integration effort.

The Organization Compatibility Test disclosed herein quantitatively measures an individual's attitude towards the factors involved in effectively performing the duties required by a given position within an organization. The OCT significantly contributes to optimized hiring since the test will objectively measure the compatibility of an individual and an organization's structure, culture and overall work environment.

In an example it is determined that in order for an individual to be a successful match with a given organization that the individual should possess the following overall characteristics in order to be a good tit for the position offered:
An attitude of “whatever it takes”;
An ability to handle and thrive in “conflict”;
The desire to confront co-workers regardless of rank in order to further his or her ideas;
Being able to take and give out criticism, regardless of how harsh it may appear to be;
In general, a very competitive nature;
The ability to work as long as necessary, including nights and weekends;
A keen, unwavering interest in data and data analysis; and
The ability to sacrifice important aspects of one's life when the job requires it.

If the pay offered by the company is high enough, it is expected that individuals who do not possess the characteristics listed above would apply for the position. Due to a variety of reasons (monetary, location, etc), when asked questions about their attitude towards the company's philosophy, the potential hire may pretend compatibility when in reality they are aware that they are not particularly comfortable in that type of environment. And if they are otherwise well-qualified, the individual may be hired and trained, only to be terminated or leave due to an incompatibility with the position's overall requirements.

The Organization Compatibility Test (OCT) aims to resolve that by addressing the issue of the individual's compatibility with a given organization in a rigorous, quantitative manner. In describing this test, a corporation will be used as an example of an organization. In practice, this test can be adapted for use in the context of almost any organizational network.

In preparation for the test, the organization's structure and environment must be mapped out from the top (the overall philosophy of the company) to the bottom (a description characterizing the environment on the level of each department or work group). Each level of description should be outlined by the individual(s) most responsible for the operations on that level. So the overall company philosophy would most likely be drafted by the President or CEO and the department descriptions would be drafted by the managers in charge of that department. Once the descriptive “map” of the organization is created, each aspect is converted into both a visual image and an auditory message containing a voice reading the respective text. The visual image would consist of the written out text as well as a visual representation(s) of the text. For example, the text representing “the ability to work as long as necessary, including nights and weekends” could be accompanied by an image of a person alone at his desk in a dimly lit office concentrating on his or her work. After these materials are assembled, the OCT may be performed.

Attitude assessment and EEG based qualification of emotion

### Physiology

Neurologically, the limbic system, which is also involved in motivation and memory processing, is responsible for the initial emotional interpretation of a given stimulus. The processed signal is then sent to the hypothalamus which analyzes it further and triggers an appropriate physical response (increased heart rate for fear, sweating for anxiety, etc). The signal then travels to the amygdala where it is associated with a template of emotional reactions such as reward or fear, and compared to previous experiences before going on to further processing in the cortex. Since the limbic system is located within the brain, the progression just described cannot be detected through electrical means on the scalp.

After the limbic system, however, the signal travels to the temporal and prefrontal cortices where the visceral sensations described herein are processed on a cognitive level.

The prefrontal lobe acts as an emotional control center, and since it is part of the outer cortex, its activity can be measured from the scalp.

Distinguishing emotions

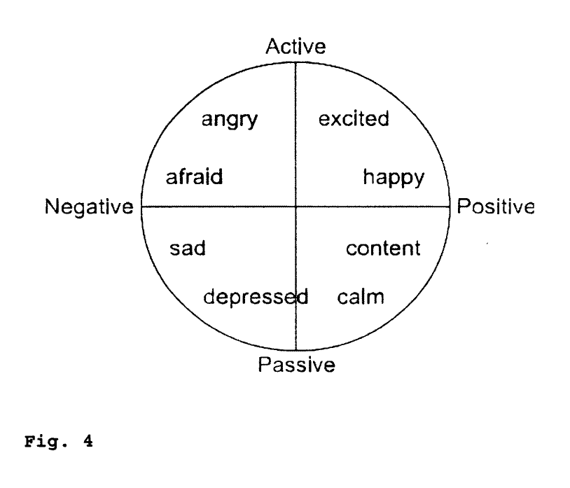

Emotional assessments are made through the use of alpha/beta frequency ratios. Alpha waves typically fall in the 8-12 Hz range and indicate a state of lower brain activity and relaxation. Beta waves fall typically within the 12-30 Hz range and indicate a state of heightened brain activity. In terms of discriminating between emotional responses, Lang's model of valence and arousal is typically used. Valence measures the nature of the emotion, whether it is positive/approach or negative/withdrawal, and arousal measures the intensity of the emotion, calm versus excited. The emotional field is divided into four quadrants as shown in FIG. 4.

When distinguishing where a particular emotional response should be placed, both alpha and beta frequencies are analyzed. The larger the proportion of alpha to beta waves, the less aroused the emotional response. Hence, a high alpha/beta ratio would place the emotional response in the lower two quadrants. Left prefrontal lobe inactivation is a sign of a negative/withdrawal response while right prefrontal lobe inactivation is a sign of a positive/approach response. Hence if a bilateral electrode was set up over the two hemispheres of the brain, a higher alpha/beta ratio over the left lobe would indicate a negative emotional response while a higher alpha/beta ratio over the right lobe would indicate a positive emotional response. These measurements determine the valence, and taken together with arousal, would place the emotional response into one of the four quadrants. One issue with this approach is that these readings do not so much qualify affective valence (the feeling of a particular emotion), but a motivational direction, namely whether the subject wants to approach or withdraw from the stimulus. For the most part, this method will generate results that correspond to affective valence. Care must be taken with the emotion of anger. Anger would indicate a low alpha/beta ratio, meaning high brain activity, but would also indicate a higher alpha/beta ration over the right lobe rather than the left since the person wants to approach and remove the stimulus rather than withdraw from it. Since the OCT is primarily interested in motivational direction, Lang's model is sufficient for its requirements. Moreover, anger is usually easily detected by an observer and the angry subject's test results can be disregarded.

### Example Protocol:

The OCT begins with the potential hire coming in to be interviewed. Once the interviewer finishes going over the individual's qualifications, the potential hire will put on a wireless EEG headset with two active sensors. First, their EEG recordings will be analyzed to produce a general frequency band layout. Principle component analysis (PCA) will be used to calculate each ratio addressed in this document. An alpha/beta frequency ratio will be calculated in order to determine whether the subject is alert enough for the test. A ratio above a certain threshold would mean that the subject is not alert and he or she will have to come back for testing at a later time.

Next, two emotionally neutral images will be displayed. The images will be taken from the emotion annotated image library (IAPS), a database of visual images whose emotional responses have already been determined over an extensive population study. These neutral images will act as the baseline frequencies for both alpha and beta frequency bands indicating a neutral motivational direction. The subject will then be presented with components of the organizational map compiled earlier, corresponding to the position that they are interviewing for. Hence, even though every interviewer will be presented with the components found at the top of the map (the company's philosophy), the rest of the stimuli will stem from the branches corresponding to the particular department where the position is located. Each visual image (text with illustration) will be accompanied by a matching auditory message. The baseline (motivation neutral frequencies) is then subtracted from the recorded frequency bands and the alpha/beta ratios will be calculated accordingly. The method for classifying emotion described above will then be applied.

At the end of the protocol, each component of the organizational map will have the potential hire's corresponding attitude assessment. Based on that data, the manager in charge of hiring will decide whether the individual is a good fit for the particular job. Along with the person's verifiable qualifications, the OCT test will allow the hiring manager (or whoever is responsible for hiring) to determine the overall compatibility of the individual with the company, thereby optimizing the organizational network with each new hire.

EEG recording and the apparatus used therefor are described in Allison et al., U.S. Patent Application Publication No. 2005/0017870; Preston, U.S. Pat. No. 5,267,570; Gevins, U.S. Pat. No. 5,724,987; Gevins, U.S. Pat. No. 5,447,166; Gevins, U.S. Pat. No. 5,295,491; Maynard, U.S. Pat. No. 5,816,247; Burton, U.S. Patent Application Publication No. 2004/0044293; Levendowski et al., U.S. Pat. No. 6,625,485; Levendowski et al., U.S. Pat. No. 6,496,724; Johnson, U.S. Pat. No. 6,754,524; Moore-Ede, U.S. Pat. No. 6,511,424; Moore-Ede, U.S. Pat. No. 6,070,098; and Pavelka., WO 2006/000166, each of which is hereby incorporated by reference.

Traditionally, an EEG was recorded using hollow disk electrodes made from tin, silver or gold. The electrodes were attached to the subject's scalp using conduction paste in order to minimize noise and impedance of the signal. The subject's scalp had to be prepared by cleansing the areas involved in the experiment usually through abrasion. Recently, a new type of electrode has been developed that functions through an active setup. The electrode is able to tolerate high levels of impedance and consequently prior skin preparation is no longer necessary. The new electrode, available as for example the BioSemi Pin-Type active electrode, contains an Ag-AgCl tip which eliminates most noise and significantly lowers signal impedance. The electrode is fitted into specially designed holders on the BioSemi headcap which are filled with electrode gel through a syringe. The elastic headcap is then fitted atop the subjects head and the EEG data collection can begin. The technology disclosed herein can employ the active electrode setup so as to minimize time and participant discomfort. After the electrode holders are filled with gel and the appropriate electrodes are attached, the electroencephalogram of many individuals can be obtained without any further setup. The individual in charge of running the technology replaces the electrode gel as needed. Wireless EEG headsets further reduce discomfort.

In order to record EEG, a minimum of two electrodes is necessary. One electrode must be placed at the reference point and another at the site of interest. The reference point should be electrically neutral so as to act as a baseline (different from the pre-signal baseline used to measure ERPs) which coupled with the signal from the electrode on the scalp will be used to calculate the EEG voltage potential readings. Typically the mastoids or the ears are used as the reference point: the mastoids being well insulated by a particularly thick layer of bone to impede the signal and the ears being far enough from the signal source to pick up anything substantial. In the present case bilateral electrodes are used so as to differentiate between left cerebral hemisphere and right cerebral hemisphere activity.

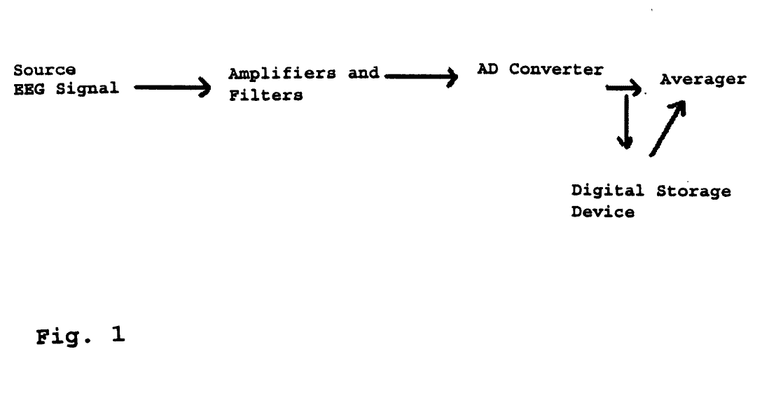

FIG. 1 describes the path of the raw EEG signal as it is converted into a form that is usable for analytical purposes. The signal is first passed through amplifying and filtering systems which increase the strength of the signal, accentuate the desired portions and filter out any unwanted frequencies. The gain should be set high enough so that the amplitude is sufficiently sensitive to pick up small deflections, but low enough so that saturation or clipping does not occur. The filtering system should couple a low pass and high pass filter in order to control for noise or artifacts. The modified signal is then sent to an Analog to Digital Converter (A/D Converter) which samples the analog signal, typically at 100 Hz, and converts the data into a digital stream. The EEG recording is now usable for software analysis. Applying a Fast Fourier Transform (FFT) at this point decomposes the complex signal into its underlying sine wave constituents, and a frequency band diagram can be composed that illustrates the prominence of different frequencies in the subject's EEG recording. An electroencephalogram can be decomposed into frequency bands which can then be analyzed to determine the person's attentive state using power ratio component analysis for example.

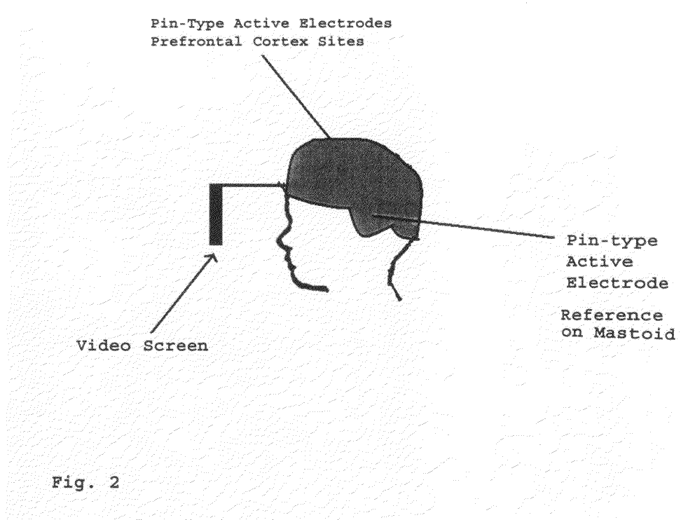

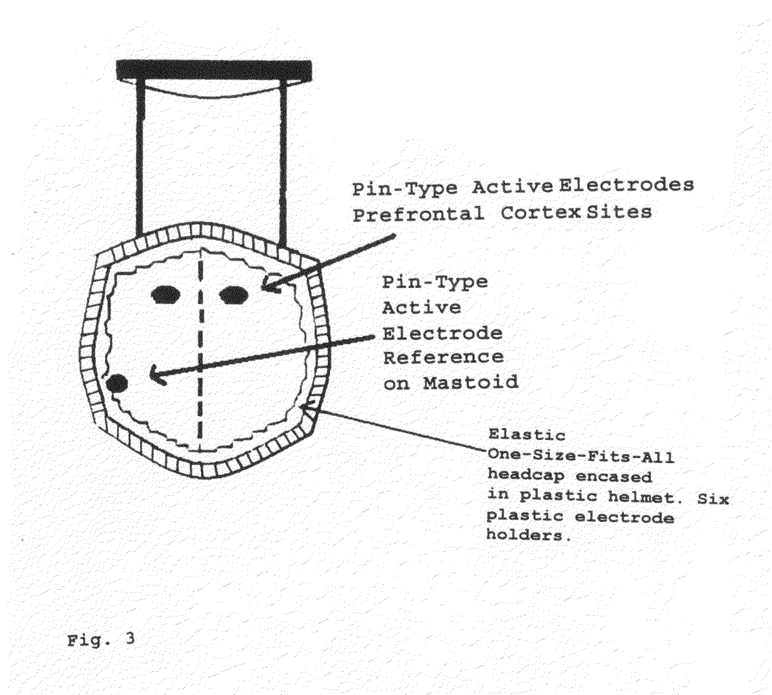

The elements that receive and modify the raw EEG signal can be effectively implemented in the current state. The technology that collects the actual EEG signal on the other hand can be modified in order to meet the requirements mentioned above. A design for such device is depicted in FIGS. 2 and 3.

The outside of the device consists of a plastic helmet measuring between 62-66 cm in circumference, able to fit most individuals since head circumferences typically range between 46-62 cm. The helmet has 3 holes for the electrode holders, allowing them to be filled periodically with electrical gel by the person who maintains it. As mentioned above, the use of active electrodes eliminates the need for scalp cleansing and therefore cuts the prep time for EEG recoding to essentially nothing. The 2 electrode holders at the top of the helmet are meant for the recording of EEG signals from the prefrontal cortex. One electrode is placed over the left hemisphere of the prefrontal cortex (LHC) and the other electrode is placed over the right hemisphere of the prefrontal cortex (RHC). The third electrode is placed over the mastoid and will serve as an EEG signal reference.

The inside of the helmet contains an elastic headcap, similar to the one designed by BioSemi, to which the electrode holders are actually attached. It will comfortably fit on the heads of most individuals and allow for maximal proximity of the electrodes to the scalp. A video screen can be attached to the front of the helmet to display the appropriate visual stimuli. It can work in conjunction with the EEG recording software and presents stimuli according to the conditions set by the individual running the technology.

### EXAMPLES

An individual is attached to an electroencephalograph apparatus for recording (EEG) over a first period of time and it is determined from the EEG that the subject is alert enough to proceed with the testing. Using a bilateral electrode with one pole of the bilateral electrode positioned over the left cerebral hemisphere (LCH) of the test subjects' brain and with another pole of the bilateral electrode positioned over the right cerebral hemisphere (RCH) of the test subjects' brain, the individual is exposed to a series of two visual images, which images have been predetermined to be emotionally neutral in a reference population of subjects (from IAPS), and the tester quantitates the levels of alpha and beta wave activity in the individual subject so as to determine baseline alpha and beta wave activity levels. Then a third EEG is recorded over a third period of time from the test subject and the test subject is exposed during the third period of time to a series of pairs of sensory stimuli, wherein the pairs each consist of a visual stimulus and a corresponding auditory stimulus, and the tester quantitates the levels of alpha and beta wave activity during exposure of the subject to the series of pairs of sensory stimuli. The baseline levels of alpha and beta wave activity are subtracted from the levels of alpha and beta wave activity, respectively, quantitated during exposure of the subject to the series of pairs of sensory stimuli so as to determine corrected alpha wave activity and corrected beta wave activity in the subject. The ratio of corrected alpha wave activity to corrected beta wave activity is determined and it is seen if the ratio of corrected alpha wave activity to corrected beta wave activity is higher in the RCH of the test subject or in the LCH of the test subject. A higher alpha/beta ratio in the RCH than the LCH is found indicating that the test subject has an attitude compatible with employment at the predetermined organization.

An individual is attached to an electroencephalograph apparatus for recording (EEG) over a first period of time and it is determined from the EEG that the subject is alert enough to proceed with the testing. Using a bilateral electrode with one pole of the bilateral electrode positioned over the left cerebral hemisphere (LCH) of the test subjects' brain and with another pole of the bilateral electrode positioned over the right cerebral hemisphere (RCH) of the test subjects' brain, the individual is exposed to a series of two visual images, which images have been predetermined to be emotionally neutral in a reference population of subjects (from IAPS), and the tester quantitates the levels of alpha and beta wave activity in the individual subject so as to determine baseline alpha and beta wave activity levels. Then a third EEG is recorded over a third period of time from the test subject and the test subject is exposed during the third period of time to a series of pairs of sensory stimuli, wherein the pairs each consist of a visual stimulus and a corresponding auditory stimulus, and the tester quantitates the levels of alpha and beta wave activity during exposure of the subject to the series of pairs of sensory stimuli. The baseline levels of alpha and beta wave activity are subtracted from the levels of alpha and beta wave activity, respectively, quantitated during exposure of the subject to the series of pairs of sensory stimuli so as to determine corrected alpha wave activity and corrected beta wave activity in the subject. The ratio of corrected alpha wave activity to corrected beta wave activity is determined and it is seen if the ratio of corrected alpha wave activity to corrected beta wave activity is higher in the RCH of the test subject or in the LCH of the test subject. A higher alpha/beta ratio in the LCH than the RCH is found indicating that the test subject does not possesses an attitude compatible with employment at the predetermined organization

1. A method of determining whether a test subject possesses an attitude compatible with employment at a predetermined organization comprising:
a) recording a first electroencephalograph (EEG) from the test subject over a first period of time and performing a power component analysis on brain wave activity from the EEG of the test subject so as to thereby determine if the subject is alert enough to proceed to step b) of the method, and if so, proceeding to step b);
b) recording a second EEG from the test subject over a second period of time and exposing the test subject during the second period of time to a series of visual images, which images have been predetermined to be emotionally neutral in a reference population of subjects, and quantitating the levels of alpha and beta wave activity in the test subject during the second period of time so as to determine baseline alpha and beta wave activity levels;
c) recording a third EEG over a third period of time from the test subject and exposing the test subject during the third period of time to a series of pairs of sensory stimuli, wherein the pairs each consist of a visual stimulus and a corresponding auditory stimulus, and quantitating the levels of alpha and beta wave activity in the test subject during exposure of the subject to the series of pairs of sensory stimuli;
d) subtracting the baseline levels of alpha and beta wave activity quantitated during step b) from the levels of alpha and beta wave activity, respectively, quantitated during step d), so as to determine corrected alpha wave activity and corrected beta wave activity in the subject;
e) determining the ratio of corrected alpha wave activity to corrected beta wave activity,
f) determining if the ratio of corrected alpha wave activity to corrected beta wave activity is higher in the RCH of the test subject or in the LCH of the test subject,
wherein a higher alpha/beta ratio in the RCH than the LCH indicates that the test subject has an attitude compatible with employment at the predetermined organization, and wherein a higher alpha/beta ratio in the LCH than the RCH indicates that the test subject does not possesses an attitude compatible with employment at the predetermined organization.

2. The method of claim 1 wherein (1) a higher alpha/beta ratio in the RCH than LCH and an alpha/beta ratio in excess of two standard deviations of baseline indicates that the subject is content and/or calm and possesses an attitude compatible with the predetermined organization, and (2) a higher alpha/beta ratio in the RCH than LCH and an alpha/beta ratio below two standard deviations of baseline indicates that the subject is excited and/or happy and possesses an attitude compatible with the predetermined organization.

3. The method of claim 1, wherein the first, second and third EEGs are recorded using a bilateral electrode with one pole of the bilateral electrode positioned over the left cerebral hemisphere of the test subjects' brain and with another pole of the bilateral electrode positioned over the right cerebral hemisphere of the test subjects' brain.

4. The method of claim 1, wherein in step a) the test subject is determined to be alert by (a) an alpha waveband power component ratio of 0.5-1.0 and a theta waveband power component ratio of less than 0.5 or (b) a theta waveband power ratio of 0.5-1.0 and an alpha waveband power component ratio of less than 0.5.

5. The method of claim 1, wherein the EEGs are recorded using electrodes each comprising an Ag-AgCl recording tip.

6. The method of claim 1, wherein a 3-D reconstruction of the EEG recorded is not performed.

7. The method of claim 1, wherein no p300 recordings are made from the subject during the first, second and third period of time.

8. The method of claim 1, wherein the pairs of sensory stimuli consisting of a visual stimulus and a corresponding auditory stimulus are provided by the predetermined organization.

9. The method of claim 1, wherein the EEGs are recorded by a wireless EEG headset.

10. The method of claim 1, wherein in step b) the test subject is exposed to a series of two visual images.

11. The method of claim 1, wherein in step c) the visual image of the pair of stimuli comprises text and an illustration.

12. A method of determining whether a test subject possesses an attitude compatible with performing work for a predetermined organization comprising:
a) recording a first electroencephalograph (EEG) from the test subject over a first period of time and performing a power component analysis on brain wave activity from the EEG of the test subject so as to thereby determine if the subject is alert enough to proceed to step b) of the method, and if so, proceeding to step b);
b) recording a second EEG from the test subject over a second period of time and exposing the test subject during the second period of time to a series of visual images, which images have been predetermined to be emotionally neutral in a reference population of subjects, and quantitating the levels of alpha and beta wave activity in the test subject during the second period of time so as to determine baseline alpha and beta wave activity levels;
c) recording a third EEG over a third period of time from the test subject and exposing the test subject during the third period of time to a series of pairs of sensory stimuli, wherein the pairs each consist of a visual stimulus and a corresponding auditory stimulus, and quantitating the levels of alpha and beta wave activity in the test subject during exposure of the subject to the series of pairs of sensory stimuli;
d) subtracting the baseline levels of alpha and beta wave activity quantitated during step b) from the levels of alpha and beta wave activity, respectively, quantitated during step d), so as to determine corrected alpha wave activity and corrected beta wave activity in the subject;
e) determining the ratio of corrected alpha wave activity to corrected beta wave activity,
f) determining if the ratio of corrected alpha wave activity to corrected beta wave activity is higher in the RCH of the test subject or in the LCH of the test subject,
wherein a higher alpha/beta ratio in the RCH than the LCH indicates that the test subject has an attitude compatible with performing work for the predetermined organization, and wherein a higher alpha/beta ratio in the LCH than the RCH indicates that the test subject does not possesses an attitude compatible with performing work for the predetermined organization.

13. The method of claim 12, wherein no p300 recordings are made from the subject during the first, second and third period of time.

14. The method of claim 12 wherein (1) a higher alpha/beta ratio in the RCH than LCH and an alpha/beta ratio in excess of two standard deviations of baseline indicates that the subject is content and/or calm and possesses an attitude compatible with performing work for the predetermined organization, and (2) a higher alpha/beta ratio in the RCH than LCH and an alpha/beta ratio below two standard deviations of baseline indicates that the subject is excited and/or happy and possesses an attitude compatible with performing work for the predetermined organization.

15. The method of claim 12, wherein the first, second and third EEGs are recorded using a bilateral electrode with one pole of the bilateral electrode positioned over the left cerebral hemisphere of the test subjects' brain and with another pole of the bilateral electrode positioned over the right cerebral hemisphere of the test subjects' brain.

16. The method of claim 12, wherein in step a) the test subject is determined to be alert by (a) an alpha waveband power component ratio of 0.5-1.0 and a theta waveband power component ratio of less than 0.5 or (b) a theta waveband power ratio of 0.5-1.0 and an alpha waveband power component ratio of less than 0.5.

17. The method of claim 12, wherein the EEGs are recorded using electrodes each comprising an Ag-AgCl recording tip.

18. The method of claim 12, wherein a 3-D reconstruction of the EEG recorded is not performed.

19. The method of claim 12, wherein the pairs of sensory stimuli consisting of a visual stimulus and a corresponding auditory stimulus are provided by the predetermined organization.

20. The method of claim 12, wherein the EEGs are recorded by a wireless EEG headset.

21. (canceled)

22. (canceled)
